(19) United States
(12) Patent Application Publication
(10) Pub. No.: US 2023/0285715 A1
(43) Pub. Date: Sep. 14, 2023

(54) IMAGING PLACED CATHETER

(71) Applicant: Mohammad Nasser, Phoenix, AZ (US)

(72) Inventor: Mohammad Nasser, Phoenix, AZ (US)

(21) Appl. No.: 18/119,326

(22) Filed: Mar. 9, 2023

Related U.S. Application Data
(60) Provisional application No. 63/318,115, filed on Mar. 9, 2022.

(51) Int. Cl. A61M 25/00

(52) U.S. Cl. CPC A61M 25/0017; A61M 2025/0166

(57) ABSTRACT

An imaging placed catheter comprising a tubular member made of one or more flexible sanitary materials with an imaging device removably coupled to the tubular member. A stopper is adjustably connected to the tubular member allowing the stopper to be selectively moved and secured along the axis of the tubular member at a desire position. One or more supports are connected to the stopper, and adjustably connected to a belt. The belt and supports secure the stopper in a desired position. A leg strap is optionally connected to the tubular member to secure the tubular member to a patient's leg, keeping the tube from catching on miscellaneous objects. The stopper may be comprised of a disc and a clamp. The clamp is any clamp that holds the disc in place without impeding the flow or urine through the tubular member.

### CROSS REFERENCE TO RELATE APPLICATION

This application claims benefit from currently pending U.S. Provisional Application No. 63/318,115 titled “IMAGING PLACED CATHETER” and having a filing date of Mar. 9, 2022, all of which is incorporated by reference herein.

### TECHNICAL FIELD

The present specification relates generally to catheters, and more particularly to urethral catheters placed with assistance from an imaging method.

### BACKGROUND

Catheters are tube like devices used to drain urine from a patient's bladder. For many years it has been common practice to utilize a catheter for the drainage of urine from the bladder of infirm or disabled individuals who are unable to empty their bladder in a normal fashion. A common catheter is a Foley catheter which includes a balloon which is associated with the insertion end of the catheter and which may be inflated larger than the diameter of the urethra for retaining the catheter in the bladder. The insertion end of the catheter tube ends above the balloon, which prevents the patient's bladder from being completely drained. The balloon allows for the catheter to remain in place in the bladder for an extended period following insertion. Thus, Foley catheters are also known as “indwelling” catheters because they are designed to be left in place for a period of time. The urine that remains in the bladder due to the end of the catheter tube being above the balloon can increase the patient's risk of a bladder infection.

### SUMMARY

Accordingly, the present catheter has been made in view of the above-mentioned disadvantages occurring in the prior art. One advantage of the catheter is to allow for consistent placement of the catheter's insertion end at the base of the bladder, proximal to the internal urethral sphincter which allows for more urine to be drained, greater comfort for the patient, and decreased risk of bladder infection.

A male catheter to be used on a patient may comprise a tubular member. The tubular member may be comprised of one or more flexible sanitary materials, such as latex and the like. The catheter may comprise an imaging device, such as, for example, a camera which is removably coupled to the tubular member to assist in the placement of the catheter insertion end, and then be removed. The catheter may comprise a stopper. The stopper may be adjustably connected to the tubular member such that the stopper may be selectively moved and secured along the axis of the tube at a desired position. The stopper may comprise a disc and clamp. The clamp may be any clamp that will hold the stopper in the desired position without impeding the flow of urine through the tube, such as a friction clamp or the like. The catheter may also comprise a leg strap that may be adjustably connected to the tube.

A method of using the catheter on a patient who has a penis, the method comprising placing the tubular member in the urethra. Viewing the interior of the urethra, the bladder, and the internal urethral sphincter with an imaging device. Using one or more images from the imaging device to place the insertion end of the tubular member at the base of the bladder, proximal to the internal urethral sphincter. Removing the imaging device from the tubular member. Adjusting a stopper proximal to the head of the penis. The stopper may prevent the tubular member from moving into the bladder. The stopper may have one or more supports. Placing a belt around the waist of the patient. Adjusting the one or more supports to comfortably attach to the belt. The supports may prevent the catheter tubular member from moving away from the bladder. The combination of the stopper and the support straps may keep the insertion end of the catheter tube at the base of the bladder, proximal to the internal urethral sphincter. Attaching a leg strap around the leg of the patient. The leg strap may be connected to the catheter tubular member.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the. Absent such clear statements of intent to apply a “special” definition, it is the inventor's intent and desire that the simple, plain, and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for” and will also recite the word “function” (i.e., will state “means for performing the function of . . . , without also reciting in such phrases any structure, material or act in support of the function. Thus, even when the claims recite a “means for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 is an isometric view of a catheter in accordance to one, or more embodiments;

FIG. 2 is a side view of an imaging device in a catheter tube in accordance to one, or more embodiments;

FIG. 3 is a front view of a stopper separate from a clamp in accordance to one, or more embodiments; and

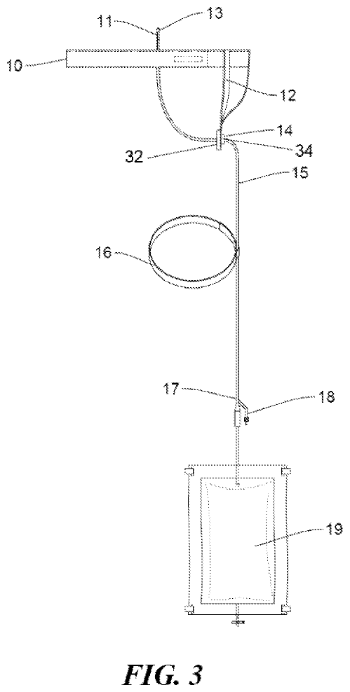

FIG. 4 is an isometric view of a catheter placed on a male patient in accordance to one, or more embodiments.

### DETAILED DESCRIPTION

This disclosure relates to an imaging placed catheter. The catheter insertion end is placed at the base of the bladder with the assistance of an imaging device to determine correct placement. One advantage provided by the imaging placed catheter may be a reduction in occurrences of bladder infections in the patient. The imaging placed catheter is described here as being placed in a patient with a penis, but it may also be used with patients without a penis, such as, for example, female patients.

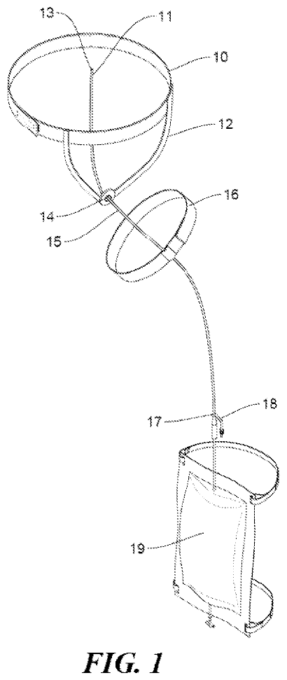

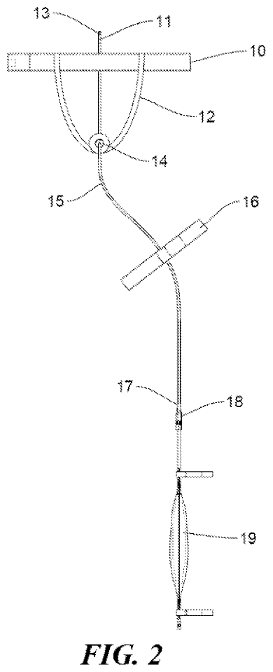

Referring initially to FIG. 1-2, the isometric view of an embodiment of an imaging placed catheter is shown generally at 8. The imaging placed catheter 8 may comprise a tubular member 15 with an insertion end 13 and a distal end 17. The tubular member 15 may be comprised of one or more flexible sanitary materials, such as, for example, latex, silicone, polyurethane, polyisoprene, lambskin, nitrile, rubber, thermoplastic elastomers, thermoplastic polymers, and the like. An imaging device 11 can be, such as, for example, a video camera, a still camera, compact camera, endoscope, microscopic camera, or the like. The imaging device 11 may be removably coupled to the interior of the tubular member 15 substantially near or at the insertion end 13 wherein the tubular member can have an O-ring on the inner diameter that can hold the imaging device in place, or imaging device can be removeable attached to the tubular member by press fitting the inner diameter of the tubular member around the imaging device. In other embodiments, the imaging device 11 can have an O-ring wherein the tubular member can have an O-ring groove on its inner diameter wherein the O-ring can sit within the groove and keep the imaging device stationary in the tubular member.

In embodiments, a stopper 14 may be adjustably connected to the tubular member 15 by such as, for example, a clamp, a retaining ring, tube clamp, elastic bands, or the like. The stopper 14 may be selectively moved along the axis of the tubular member 15 and once in the desired position the stopper may be secured to the tubular member. One or more supports 12 may be adjustably connected to the stopper 14 and a belt 10 by such as, for example, a buckle, a hinged bar, a feed through cam, a squeeze release, or the like. The supports 12 can be adjusted in length to allow for different size users and to allow for different size penis lengths. The belt 10 may be secured around the waist of the patient. A leg strap 16 may be connected to the tubular member 15 in between the distal end 17 and the stopper 14. The distal end 17 of the tubular member 15 comprises a connector 18 that is configured to accept a urine bag 19. The urine bag 19 and connector 18 may be configured so that the urine bag 19 can be easily replaced.

The stopper 14 may be comprised of a disc 32 and a clamp 34. The disc 32 and the clamp 34 may be formed as one piece, or they may be separate pieces. The disc 32 may be any shape, such as, for example, circular, elliptical, square, or the like. The clamp 34 may be any type of clamp that will hold the stopper 14 or the disc 32 in the desired position without impeding the flow of urine through the tubular member 15, such as, for example, a friction clamp, a tube clamp, a hose clamp, worm clamp, attachment clamp, and the like. The stopper disc 32 may have one or more slots (not shown) substantially near the outer edges of the stopper disc wherein the supports 12 can be adjustably attached to the stopper disc through the slots. In certain embodiments the slots can be omitted and the supports 12 can be attached directly to the stopper disc 32 or the stopper disc can have one or more protrusions on its outer surface wherein the protrusions can have slots that the supports can be adjustably attached to.

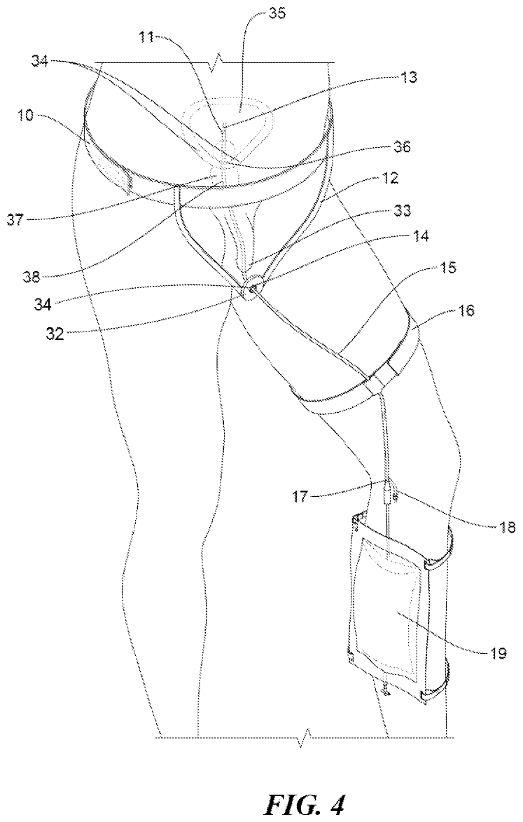

Referring now to FIG. 4, an isometric view of the catheter in a patient. In use the insertion end 13 of the tubular member 15 is inserted into the patient's urethra 38. The imaging device 11 is used to view the urethra 38, bladder 35, and internal urethral sphincter 34 of the patient. Using one or more images from the imaging device 11, the insertion end 13 of the tubular member 15 is placed at the base 36 of the bladder 35, proximal to the internal urethral sphincter 34. This placement of the insertion end 13 allows for the bladder 35 to drain more completely, thus reducing the risk of infection to the patient. Once the insertion end 13 of the tubular member 15 is in the desired location, the stopper 14 is moved along the axis of the tubular member 15 and placed proximal to the end of the urethra. In the case of a patient with a penis, the placement is proximal to the head of the penis 33. The stopper 14 is then secured in the desired location. The stopper 14 prevents the tubular member 15 from moving farther into the patient and entering the bladder 35. The belt 10 can be placed around the patient about the waist. The supports 14 are adjusted so that they help to keep the stopper 12 in place. The supports 14 may help to keep the stopper 12 from moving away from the penis head 33, and thus may keep the insertion end 13 from moving away from the bladder. The leg strap 16 may be secured around the leg of the patient and may help to keep the tubular member 15 from catching on miscellaneous objects and being pulled out of place. The connector 18, as shown FIG. 1, at the distal end 17 of the tubular member 15 may be connected to a urine bag 19 and may be configured to allow for easy changing of the urine bag 19.

In embodiments, a method of using the catheter on a patient who has a penis, the method may comprise at step 1, placing the tubular member in the urethra. Step 2, viewing the interior of the urethra, the bladder, and the internal urethral sphincter with an imaging device. Step 3, Using one or more images from the imaging device to place the insertion end of the tubular member at the base of the bladder, proximal to the internal urethral sphincter. Step 4, removing the imaging device from the tubular member. Step 5, adjusting a stopper proximal to the head of the penis. The stopper may prevent the tubular member from moving into the bladder. The stopper may have one or more supports. Step 6, placing a belt around the waist of the patient. Step 7, adjusting the one or more supports to comfortably attach to the belt. The supports may prevent the catheter tubular member from moving away from the bladder. The combination of the stopper and the support straps may keep the insertion end of the catheter tube at the base of the bladder, proximal to the internal urethral sphincter. Step 8, attaching a leg strap around the leg of the patient. The leg strap may be connected to the catheter tubular member.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

What is claimed is:

1. A male catheter to be used on a patient comprising:
a tubular member having an insertion end and a distal end wherein a imaging device is removably coupled to the insertion end;
a stopper adjustably connected to the tubular member such that the stopper can be selectively moved and secured along the axis of the tubular member at a desired position; and
a belt with one or more supports connecting the belt to the stopper to secure the stopper in the desired position.

2. The catheter of claim 1, wherein a leg strap is adjustably connected to the tubular member.

3. The catheter of claim 1, wherein the tubular member is comprised of one or more flexible sanitary materials.

4. The catheter of claim 1, wherein the tubular member is comprised of latex.

5. The catheter of claim 1, wherein the stopper is comprised of a disc and a clamp.

6. The catheter of claim 4, wherein the clamp is a friction clamp.

7. A method of using a catheter on a patient having a penis, the method comprising:
placing a tubular member in the urethra
viewing the urethra, bladder, and internal urethral sphincter with an imaging device that is removably connected inside the tubular member;
placing, with assistance from the imaging device, the tubular member at the base of the bladder, proximal to the internal urethral sphincter
adjusting a stopper proximal to the head of the penis to prevent the tubular member from moving into the bladder, the stopper having one or more supports;
placing a belt around the waist of the patient;
adjusting the supports to comfortably attach to the belt, the supports preventing the tubular member from moving away from the bladder; and
attaching a leg strap connected to the tubular member around the leg of the patient.
